(19) United States
(12) Patent Application Publication
(10) Pub. No.: US 2026/0108176 A1
(43) Pub. Date: Apr. 23, 2026

(54) METHOD AND APPARATUS FOR PERSONALIZED STEP COUNT AND STRIDE LENGTH MEASUREMENT

(71) Applicant: SAMSUNG ELECTRONICS CO., LTD., Suwon-si (KR)

(72) Inventors: Himanshu DIDDEN, Noida (IN); Gunjan Gupta, Noida (IN); Akshay Bidlish, Noida (IN); Aril Kumar, Noida (IN); Anand Kumar Asati, Noida (IN)

(73) Assignee: SAMSUNG ELECTRONICS CO., LTD., Suwon-si (KR)

(21) Appl. No.: 19/423,447

(22) Filed: Dec. 17, 2025

(30) Foreign Application Priority Data
Aug. 25, 2023 (IN) 202311057057

(51) Int. Cl. A61B 5/11; A61B 5/00; A61B 5/06

(52) U.S. Cl. CPC A61B 5/1118; A61B 5/061; A61B 5/1123; A61B 5/725; A61B 2562/0219

(57) ABSTRACT

Provided is a method of controlling an electronic apparatus, the method including obtaining main motion data and sub motion data, obtaining synthetic data by combining the main motion data and the sub motion data, obtaining a threshold value based on a magnitude of a signal waveform of the synthetic data and a running mean of the synthetic data, identifying a plurality of critical points in the synthetic data based on the threshold value, identifying signal patterns, based on the plurality of critical points, in each axis of a plurality of axes in the main motion data of a 3-Dimension (3D) coordinate system, obtaining at least one similarity in the signal patterns based on a plurality of inter-axis features extracted from the synthetic data, obtaining a degree of symmetry between each axis of the plurality of axes based on the at least one similarity, and obtaining movement information of a user based on the degree of symmetry and the synthetic data.

### CROSS-REFERENCE TO RELATED APPLICATIONS

This application is a bypass continuation application of International Patent Application No. PCT/IB2024/057390 filed on Jul. 31, 2024, which is based on and claims priority to Indian Patent Application number 202311057057, filed on Aug. 25, 2023, the disclosures of which are incorporated by reference herein in their entireties.

### BACKGROUND

### 1. Field

The present disclosure generally relates to the field of pedometers and activity tracking devices, and more particularly relates to method and techniques for personalized step count and stride length measurement in smart devices.

### 2. Description of Related Art

Pedometers may be used in modern fitness tracking, offering users a convenient and motivating way to monitor their daily physical activity. The pedometers may be implemented in electronic apparatus like smartphones, smartwatches, and other wearable devices.

In some scenarios, the pedometers may be implemented as a mobile application (app), for example, in the smartphones. These electronic apparatus may detect and count steps taken by a user during a walking activity and provide valuable insights including one or more parameters corresponding to the walking activity of the user.

Thus, pedometers may be remarkably useful in promoting a more active lifestyle, encouraging individuals to set and achieve step-based goals. Many users may find pedometers to be highly effective in creating awareness about their daily movement patterns, leading to increased physical activity and improved cardiovascular health.

Pedometers may be implemented by user-friendly mobile applications that allow for easy data synchronization and tracking, making it simpler for the users to monitor their progress over time.

However, pedometers may be inaccurate. For example, Pedometers may count false steps and measure a distance covered by the user based on the false step counts which results in inaccurate measurement of distances.

In other examples, pedometers may count the steps of the users even when the users are travelling in a vehicle like bus, car, bicycle, etc. Also, pedometers may count the steps as a result of shaking of pedometers, hand movement e.g., arm swing, waving, or any other random movements/motions, or pedometers being placed in a perpetual motion machine.

Also, the distance and step counts measured by the pedometers may not vary depending upon user walking pattern and style.

Accordingly, Moreover, pedometers may count false steps due to unexpected motion, leading to potential discrepancies in recorded data.

Therefore, the above-mentioned limitations and problems associated with pedometers can be improved.

### SUMMARY

This summary is provided to introduce a selection of concepts in a simplified format that are further described in the detailed description of the disclosure. This summary is not intended to identify key or essential embodiments of the disclosure, nor is it intended for determining the scope of the disclosure.

According to an embodiment, a controlling method of an electronic apparatus comprises obtaining main motion data and sub motion data; obtaining synthetic data by combining the main motion data and the sub motion data; obtaining a threshold value based on a magnitude of a signal waveform of the synthetic data and a running mean of the synthetic data; identifying a plurality of critical points in the synthetic data based on the threshold value; identifying signal patterns, based on the plurality of critical points, in each axis of a plurality of axes in the main motion data of a 3-Dimension (3D) coordinate system; obtaining at least one similarity in the signal patterns based on a plurality of inter-axis features extracted from the synthetic data; obtaining a degree of symmetry between each axis of the plurality of axes based on the at least one similarity; and obtaining movement information of a user based on the degree of symmetry and the synthetic data.

The main motion data may comprise information related to a measurement of an acceleration of the electronic apparatus along the plurality of axes in the 3D coordinate system, and the sub motion data may comprise information related to a change in a rotational angle of the electronic apparatus in real-time along the plurality of axes in the 3D coordinate system.

The main motion data may be obtained from at least one main motion sensor and the sub motion data is obtained from at least one sub motion sensor.

The main motion data may comprise accelerometer data obtained from an accelerometer sensor. The sub motion data may include gyroscope data obtained from a gyroscope sensor. The signal patterns may be identified according to a motion of the electronic apparatus caused by a movement of the user.

Obtaining the movement of the user may comprise identifying steps of a plurality of users based on the synthetic data; identifying at least one fake step from the steps of the plurality of users based on the degree of symmetry; and obtaining the movement information of the user by eliminating the at least one fake step from the steps of the plurality of users.

The signal patterns may indicate a correlation between axis and energy ratio of different axes of the plurality of axes. The elimination of the at least one fake step may correspond to a removal of a random movement performed by a user or a movement that generates first signals similar to second signals that correspond to walking and running activity of the user.

The controlling method may further comprise obtaining a magnitude of a set of critical points from the plurality of critical points in the synthetic data, wherein the set of critical points is associated with a set of steps previously taken by a user; identifying a peak threshold and a valley threshold, based on the magnitude of the set of critical points; identifying a consistency between consecutive steps of the user based on each of the peak threshold and the valley threshold; and eliminating the at least one fake step based on identifying the consistency.

The controlling method may further comprise identifying a tilt angle of the electronic apparatus with respect to a horizontal plane based on the main motion data, wherein the tilt angle indicates an angle between a vertical axis and a normal vector of the electronic apparatus; identifying a posture of the electronic apparatus while the user is walking based on the tilt angle; identifying whether the posture of the electronic apparatus while the user is walking is a normal posture or an abnormal posture; and eliminating the posture of the electronic apparatus based on identifying that the posture of the electronic apparatus is the abnormal posture.

Obtaining the synthetic data may comprise correcting one or more erroneous critical points in the signal waveform of the main motion data based on the sub motion data. Correcting the one or more erroneous critical points in the signal waveform of main motion data may comprise performing, using one or more filters, a signal refining process to smooth the signal waveform of the main motion data; identifying a variation in magnitude of the signal waveform of the main motion data with respect to a magnitude of a signal waveform of the sub motion data; and adjusting the variation based on superimposition of the main motion data over the sub motion data.

Identifying the plurality of critical points in the synthetic data may comprise obtaining the magnitude of the signal waveform of the synthetic data; and identifying the plurality of critical points in the synthetic data based on the magnitude.

Obtaining the at least one similarity in the signal patterns may comprise extracting, from the synthetic data, the plurality of inter-axis features including correlation between axes and energy ratio of different axes of the plurality of axes; obtaining a plurality of statistical features based on the magnitude of the signal waveform of the synthetic data; and obtaining the at least one similarity in the signal patterns based on the plurality of inter-axis features and the plurality of statistical features.

The plurality of statistical features may include at least one of a mean value of the signal waveform, a standard deviation in the signal waveform, or a skewness and kurtosis of the signal waveform.

Identifying the signal patterns may comprise analyzing the synthetic data to check whether the plurality of critical points is within a reasonable time interval and whether a sequence of the plurality of critical points satisfies time and order constraints associated with human walking patterns, wherein the reasonable time interval corresponds to a time interval that is determined based on historical data associated with a walking activity of a user; and identifying the signal patterns based on a result of the analysis of the synthetic data.

Obtaining a user's movement information may comprise identifying at least three critical points based on the synthetic data; selecting at least two valleys and at least one peak based on the at least three critical points; obtaining a difference value between two consecutive valleys of the at least two valleys; obtaining a magnitude of acceleration corresponding to the at least one peak; and obtaining a stride length of a user based on the difference value between the two consecutive valleys and the magnitude of acceleration.

According to an aspect of an embodiment of the disclosure, an electronic apparatus may comprise at least one processor and memory storing instructions that, when executed by the at least one processor, cause the electronic apparatus to obtain main motion data and sub motion data, obtain synthetic data by combining the main motion data and the sub motion data, obtain a threshold value based on a magnitude of a signal waveform of the synthetic data and a running mean of the synthetic data, identify a plurality of critical points in the synthetic data based on the threshold value, identify signal patterns, based on the plurality of critical points, in each axis of a plurality of axes in the main motion data of a 3-Dimension (3D) coordinate system, obtain at least one similarity in the signal patterns based on a plurality of inter-axis features extracted from the synthetic data, obtain a degree of symmetry between each axis of the plurality of axes based on the at least one similarity, and obtain a user's movement information based on the degree of symmetry and the synthetic data.

### BRIEF DESCRIPTION OF DRAWINGS

These and other features, aspects, and advantages of the present disclosure will become better understood when the following detailed description is read with reference to the accompanying drawings in which like characters represent like parts throughout the drawings, wherein:

FIG. 1 illustrates an exemplary schematic diagram of the electronic apparatus, according to an embodiment of the present disclosure;

FIG. 2 illustrates components included in the module(s)/unit(s)/Engine(s), according to an embodiment of the present disclosure;

FIG. 3A illustrates a flow chart of a method implemented for eliminating one or more fake step counts, according to an embodiment of the present disclosure;

FIG. 3B illustrates a flow chart of a method implemented for eliminating one or more fake step counts, according to an embodiment of the present disclosure;

FIG. 4A illustrates an example of a graph of accelerometer data and gyroscope data, according to an example embodiment of the present disclosure;

FIG. 4B illustrates an example of a graph of accelerometer data and gyroscope data, according to an example embodiment of the present disclosure;

FIG. 5 illustrates an operation flow of the step sensing engine of FIG. 2, according to an embodiment of the present disclosure;

FIG. 6 illustrates an example of a technique of considering a single step;

FIG. 7 illustrates example postures of the user, according to an embodiment of the present disclosure;

FIG. 8 illustrates an example of detecting the plurality of peaks and valleys in the accelerometer data, according to an embodiment of the present disclosure;

FIG. 9 illustrates an operation flow of the fake step validation engine of FIG. 2, according to an embodiment of the present disclosure;

FIG. 10 illustrates a flow chart of a method implemented in the electronic apparatus for calculating a stride length (step length) of the user of the electronic apparatus;

FIG. 11 an example scenario depicting a calculated difference value between two consecutive valleys and a calculated magnitude of acceleration;

FIG. 12 illustrates an example of obtaining a step length;

FIG. 13 illustrates an example of artificial intelligence (AI) model;

FIG. 14 illustrates examples of electronic apparatus; and

FIG. 15 illustrates a flow chart of a method for controlling the electronic apparatus;

### DETAILED DESCRIPTION

Skilled artisans will appreciate that elements in the drawings are illustrated for simplicity and may not be necessarily drawn to scale. For example, the flow charts illustrate the method in terms of the most prominent steps involved to help to improve understanding of aspects of the disclosure.

Furthermore, in terms of the construction of the device, one or more components of the device may have been represented in the drawings by conventional symbols, and the drawings may show only those specific details that are pertinent to understanding the embodiments of the disclosure so as not to obscure the drawings with details that will be readily apparent to those of ordinary skill in the art having the benefit of the description herein.

It should be understood at the outset that although illustrative implementations of the embodiments of the present disclosure are illustrated below, the disclosure may be implemented using any number of techniques, whether currently known or in existence. The present disclosure should in no way be limited to the illustrative implementations, drawings, and techniques illustrated below, including the exemplary design and implementation illustrated and described herein, but may be modified within the scope of the appended claims along with their full scope of equivalents.

The term “some” as used herein may refer to as “none, or one, or more than one, or all.” Accordingly, the terms “none,” “one,” “more than one,” “more than one, but not all” or “all” may all fall under the definition of “some.” The term “some embodiments” may refer to no embodiments, to one embodiment or to several embodiments or to all embodiments. Accordingly, the term “some embodiments” may refer to “no embodiment, or one embodiment, or more than one embodiment, or all embodiments.”

Other variations are within spirit of disclosure. Thus, while disclosed techniques are susceptible to various modifications and alternative constructions, certain illustrated embodiments thereof are shown in drawings and have been described above in detail. It should be understood, however, that there is no intention to limit disclosure to specific form or forms disclosed, but on contrary, intention is to cover all modifications, alternative constructions, and equivalents falling within spirit and scope of disclosure, as defined in appended claims . . .

More specifically, any terms used herein such as but not limited to “includes,” “comprises,” “has,” “consists,” and grammatical variants thereof may not specify an exact limitation or restriction and certainly may not exclude the possible addition of one or more features or elements, unless otherwise stated, and furthermore must not be taken to exclude the possible removal of one or more of the listed features and elements, unless otherwise stated with the limiting language “must comprise” or “not to include.”

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element does not preclude there being none of that feature or element, unless otherwise specified by limiting language such as “there needs to be one or more” or “one or more element is required.”

Unless otherwise defined, all terms, and especially any technical and/or scientific terms, used herein may be taken to have the same meaning as commonly understood by one having ordinary skill in the art.

Phrases of form such as “at least one of A, B, and C,” or “at least one of A, B or C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such language is not generally intended to imply that certain embodiments require at least one of A, at least one of B and at least one of C each to be present.

Use of any and all examples, or exemplary language provided herein, is intended merely to better illuminate embodiments of disclosure and does not pose a limitation on scope of disclosure unless otherwise claimed. No language in specification should be construed as indicating any non-claimed element as essential to practice of disclosure.

It will be understood that when an element or layer is referred to as being “on,” “connected to” or “coupled to” another element or layer, it can be directly on, connected or coupled to the other element or layer, or intervening elements or layers may be present. By contrast, when an element is referred to as being “directly on,” “directly connected to,” or “directly coupled to” another element or layer, there are no intervening elements or layers present.

Use of terms such as “a” and “an” and “the” and similar referents in context of describing disclosed embodiments (especially in context of following claims) are to be construed to cover both singular and plural, unless otherwise indicated herein or clearly contradicted by context, and not as a definition of a term.

Terms such as “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

In addition, unless otherwise noted or contradicted by context, term “plurality” indicates a state of being plural (e.g., “a plurality of items” indicates multiple items). Number of items in a plurality is at least two, but can be more when so indicated either explicitly or by context. Further, unless stated otherwise or otherwise clear from context, phrase “based on” means “based at least in part on” and not “based solely on.

Terms such as “first,” “second,” “third,” “fourth,” etc. are used merely to distinguish elements from one another and thus do not denote any particular order unless an order is specifically described. For example, the term “second” may be used without using the term “first”. Also, although the terms “first,” “second,” “third,” and so on may be used herein to describe various elements, components, regions, layers and/or sections, these elements, components, regions, layers and/or sections should not be limited by these terms.

Embodiments of the present disclosure will be described below in detail with reference to the accompanying drawings.

An embodiment of the disclosure may include an electronic apparatus for eliminating one or more fake step counts. An embodiment of the disclosure may include a method that is implemented in the electronic apparatus configured to determine an actual number of steps taken by a user by examining a signal pattern of accelerometer data and gyroscope data from an accelerometer sensor and gyroscope sensor. Run-time thresholds may be applied to the signal pattern to incorporate varied walking/running patterns.

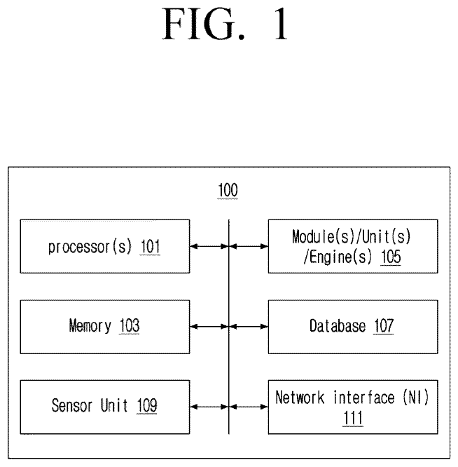

The method may include eliminating random movements and fake step counts to predict accurate unit step length, which in turn is used to calculate a personalized distance covered by the user. A detailed explanation of the same is explained in the forthcoming paragraphs corresponding to at least FIGS. 1 to 9.

FIG. 1 illustrates an exemplary schematic diagram of the electronic apparatus, according to an embodiment of the present disclosure. According to an embodiment of the present disclosure, the electronic apparatus 100 may include a processor(s) 101, a memory 103, a module(s)/unit(s)/Engine(s) 105, a database 107, a sensor unit 109, and a network interface (NI) 111. As an example, the electronic apparatus 100 may correspond to a smartwatch, a smartphone, a wearable device, or any electronic device having at least an accelerometer sensor and/or a gyroscope sensor.

In an example, the processor(s) 101 may be a single processing unit or a number of units, all of which could include multiple computing units. The processor(s) 101 may be implemented as one or more microprocessors, microcomputers, microcontrollers, digital signal processors, central processing units, logical processors, virtual processors, state machines, logic circuitries, and/or any devices that manipulate signals based on operational instructions. Among other capabilities, the processor(s) 101 may be configured to fetch and execute computer-readable instructions and data stored in the memory 103.

The memory 103 may include any non-transitory computer-readable medium including, for example, volatile memory, such as static random-access memory (SRAM) and dynamic random-access memory (DRAM), and/or non-volatile memory, such as read-only memory (ROM), erasable programmable ROM, flash memories, hard disks, optical disks, and magnetic tapes.

In an example, the module(s)/unit(s)/Engine(s) 105 may include a program, a subroutine, a portion of a program, a software component, or a hardware component capable of performing a stated task or function. As used herein, the module(s)/unit(s)/Engine(s) 105 may be implemented on a hardware component such as a server independently of other modules, or a module can exist with other modules on the same server, or within the same program. The module(s)/unit(s)/Engine(s) 105 may be implemented on a hardware component such as processor one or more microprocessors, microcomputers, microcontrollers, digital signal processors, central processing units, state machines, logic circuitries, and/or any devices that manipulate signals based on operational instructions. The module(s)/unit(s)/Engine(s) 105 when executed by the processor(s) 101 may be configured to perform any of the functionalities described herein.

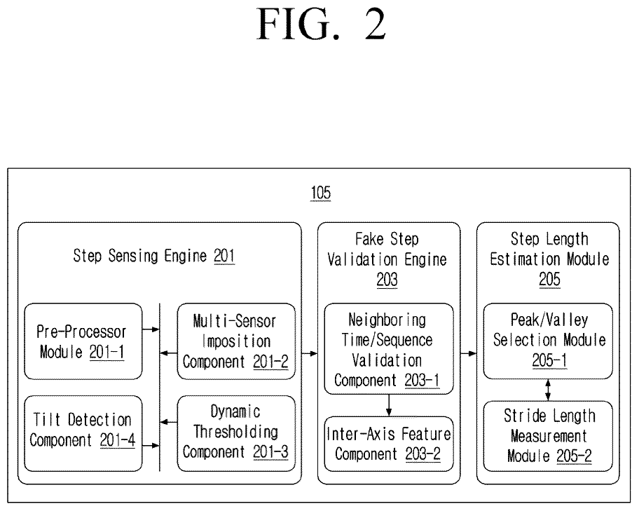

FIG. 2 illustrates components included in the module(s)/unit(s)/Engine(s), according to an embodiment of the present disclosure. According to some embodiments, the module(s)/unit(s)/Engine(s) 105 may further include a step sensing engine 201, a fake step validation engine 203, and a step length estimation module 205 coupled to each other and being operated in collaboration with each other.

The step sensing engine 201 may include a pre-processor module 201-1, a multi-sensor imposition component 201-2, a dynamic thresholding component 201-3 coupled with each other. Further, the fake step validation engine 203 may include a neighboring time/sequence validation component 203-1, an Inter-Axis Feature Component 203-2 coupled and operates in collaboration with each other.

Further, the step length estimation module 205 may include a peak/valley selection 205-1 module and a stride length measurement 205-2 module coupled and operates in collaboration with each other. According to an embodiment, the working of electronic apparatus 100 may be made through the various aforesaid components for ease of explanation. Further, according to an alternate embodiment, various operations of the components depicted in FIG. 2 can be performed by the processor(s) 101.

As a further example, the database 107 may be implemented with integrated hardware and software. The hardware may include a hardware disk controller with programmable search capabilities or a software system running on general-purpose hardware. The examples of the database 107 includes, but not limited to, in-memory databases, cloud databases, distributed databases, embedded databases, and the like. The database 107, amongst other things, may serve as a repository for storing data processed, received, and generated by one or more of the processors, and the modules/engines/units.

In an embodiment, the modules/units 105 may be implemented using one or more artificial intelligence (AI) modules that may include a plurality of neural network layers. Examples of neural networks include but are not limited to, Convolutional Neural Network (CNN), Deep Neural Network (DNN), Recurrent Neural Network (RNN), and Restricted Boltzmann Machine (RBM).

In the present disclosure, the ‘learning’ may be referred to as a method for training a predetermined target device using a plurality of learning data to cause, allow, or control the target device to make a determination or prediction. Examples of learning techniques may include, but not limited to, supervised learning, unsupervised learning, semi-supervised learning, or reinforcement learning. At least one of a plurality of AI models (e.g., CNN, DNN, RNN, RBM models) may be implemented to perform various functionality described herein through an AI model.

A function associated with an AI module may be performed through the non-volatile memory, the volatile memory, and the processor. The processor may include one or a plurality of processors. At this time, one or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU).

The one or more processors control the processing of the input data in accordance with a predefined operating rule or an AI model stored in the non-volatile memory and the volatile memory. The predefined operating rule or AI model is provided through training or learning.

As a further example, the sensor unit 109 may include sensors like an accelerometer sensor, a motion sensor, and a gyroscope sensor. As an example, the NI unit 111 establishes a network connection with a network like a home network, a public network, or a private network, and the like for connecting with the receiver devices.

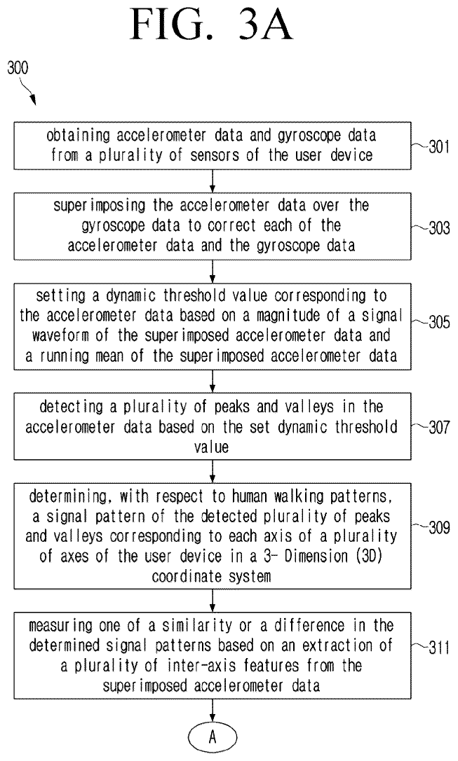

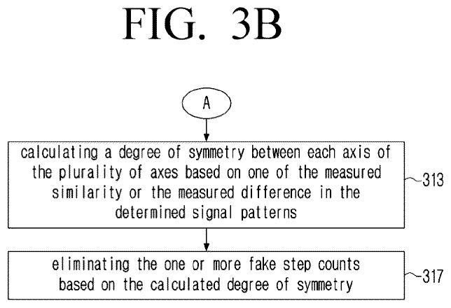

FIGS. 3A and 3B illustrate a flow chart of a method 300 implemented in the electronic apparatus 100 for eliminating one or more fake step counts, according to an embodiment of the present disclosure.

According to an embodiment, the method 300 may include a series of operation steps 301 through 317, which will be described through various components shown in at least FIGS. 2 through 10. According to one or more embodiments, each of the operation steps of the method 300 can be performed by the processor(s) 101 as shown in FIG. 1.

At step 301, the step sensing engine 201 may obtain accelerometer data and gyroscope data from a plurality of sensors of the electronic apparatus 100. As an example, the accelerometer data may be obtained from the accelerometer sensor and the gyroscope data may be obtained from the gyroscope sensor that is included in the sensor unit 109. Accordingly, the accelerometer data may include information related to a measurement of an acceleration of the electronic apparatus 100 along the plurality of axes in the 3D coordinate system namely x, y, and z.

Further, the gyroscope data may include information related to a change in a rotational angle of the electronic apparatus in real-time along the plurality of axes in the 3D coordinate system namely x, y, and z. The accelerometer sensor may measure an acceleration of the electronic apparatus 100 in 3-dimensional space namely x, y, and z.

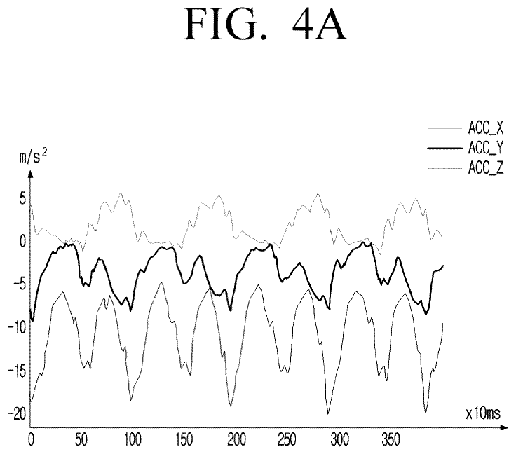

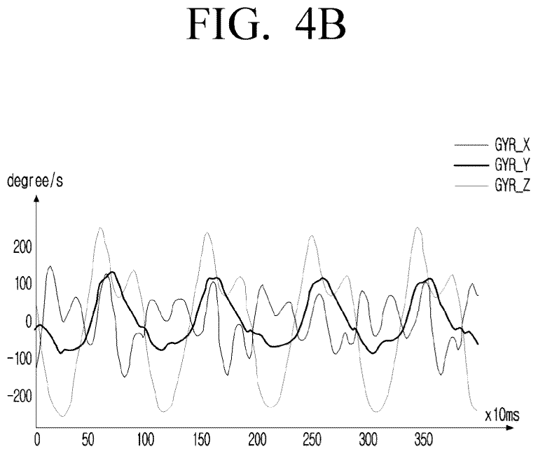

FIGS. 4A and 4B illustrate an example of a graph of accelerometer data and gyroscope data, according to an example embodiment of the present disclosure.

FIG. 4A shows an example of a graph of accelerometer data. FIG. 4B shows an example of a graph of the gyroscope data. In an example, electronic apparatus 100 can be a smartwatch. Accordingly, the step sensing engine 201 may measure the acceleration along three axes and the angular velocity of the smartwatch around three axes.

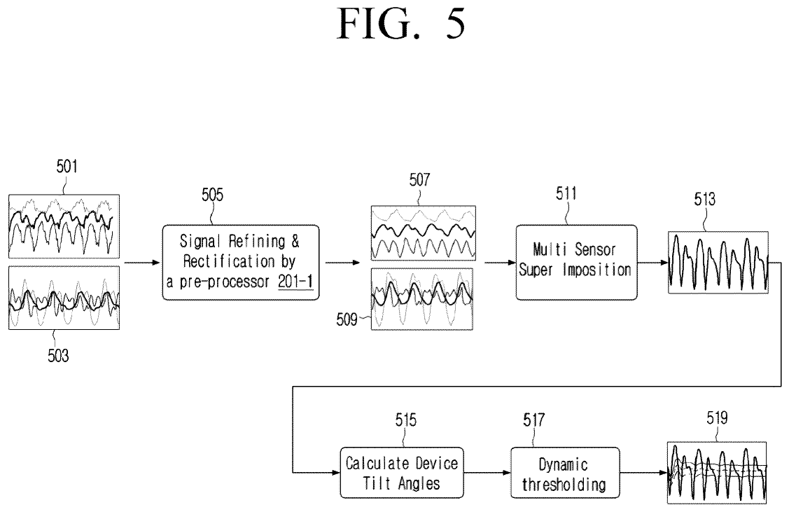

According to an embodiment, the accelerometer data and the gyroscope data may be received by the pre-processor module 201-1 for further processing. FIG. 5 illustrates an operation flow of the step sensing engine 201, according to an embodiment of the present disclosure. According to an embodiment, the pre-processor module 201-1 of the step sensing engine 201 may receive the accelerometer data 501 (referring to FIG. 4A and FIG. 5) and the gyroscope data 503 (referring to FIG. 4B and FIG. 5). In general, the accelerometer data 501 and the gyroscope data 503 may contain noise due to involuntary hand vibrations and motions during walking/running and the like.

Therefore, the pre-processor module 201-1 may remove noise from the accelerometer data 501 and the gyroscope data 503 that occurs due to involuntary hand vibrations and motions during walking/running. According to an embodiment, at block 505 signal refining may be performed to smooth out the signals of the accelerometer data 501 and the gyroscope data 503, thereby removing the noise from the signals.

According to an embodiment, the signals of the accelerometer data and the gyroscope data may be alternately referred to as accelerometer signal data/accelerometer signal waveform and gyroscope signal data/gyroscope signal waveform. This operation by the pre-processor module 201-1 may make it easier to use the sensor output for further processing. In a non-limiting example, the smoothening of the accelerometer data 501 and the gyroscope data 503 may be done using Input Infinite Response (IIR) Filter (e.g., a single Pole Low Pass IIR filter). The filtered value may be given by equation (1):

$$filtered {value}_{i} = filtered {value}_{i - 1} + ( ( 1 - decay ) ⋆ ( raw {value}_{i} - filtered {value}_{i -} \text{?} \qquad ( 1 )$$

$$\text{?} \text{indicates text missing or illegible when filed}$$

The filtered accelerometer data 507 and the filtered gyroscope data 509 may be then provided as input to the multi-sensor imposition component 201-2 of the step sensing engine 201. Though the accelerometer data 501 and the gyroscope data 503 are filtered, the filtered accelerometer data 507 and the filtered gyroscope data 509 may still contain some erroneous peaks which may hamper the actual step detection due to noise.

Thus, using superimposition/fusion of the filtered accelerometer data 507 and the filtered gyroscope data 509 may help to mitigate wrong peaks. According to an embodiment, the signals of the filtered accelerometer data and the filtered gyroscope data may be alternately referred to as filtered accelerometer signal data/filtered accelerometer signal waveform and filtered gyroscope signal data/filtered gyroscope signal waveform.

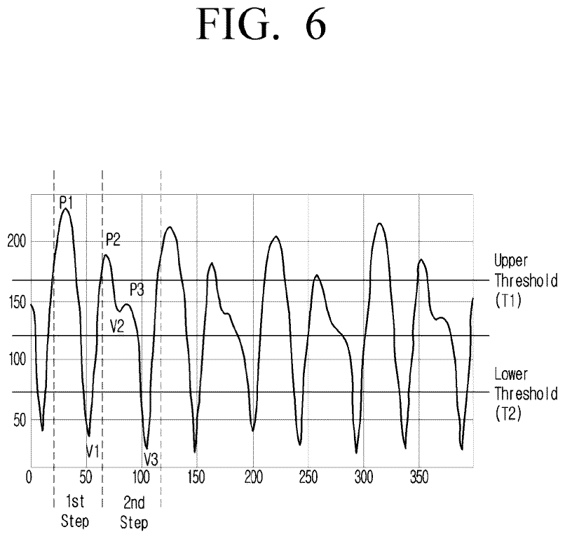

FIG. 6 illustrates an example of a technique of considering a single step. Peaks may refer to the values that are greater than their immediate neighboring (previous & next) values.

As shown in FIGS. 6, P1, P2, and P3 may correspond to peaks. Further, the valleys may refer to values that are smaller than their immediate neighboring (previous & next) values. As shown in FIGS. 6, V1, V2, and V3 may correspond to valleys. One step is counted when a pair of valid peak-valley is detected. A valid peak is defined as a peak that crosses an upper threshold (T1) and a valid valley is defined as a valley that crosses a lower threshold (T2). For a step to be counted, both peak and valley should be valid.

As shown in FIG. 6, P1-V1 and P2-V3 may be valid peak-valley pairs. Hence, they may contribute to step counted. Further, V2-P3 is may not a valid peak-valley pair as P3 does not cross upper threshold (T1) and V2 does not cross lower threshold (T2). These upper and lower thresholds can be determined statically or dynamically. Accordingly, it may be important to identify the wrong peaks and eliminate them for determining the accurate step counts.

Referring back to FIGS. 3 and 5, at step 303, the multi-sensor imposition component 201-2 of the step sensing engine 201, may superimpose the accelerometer data over the gyroscope data to correct each of the accelerometer data and the gyroscope data. In particular, the filtered accelerometer data 507 may be superimposed over the filtered gyroscope data 509 to correct each of the accelerometer data and the gyroscope data.

Thus, the corresponding components e.g., the X component, Y component, and Z component of the filtered accelerometer data 507 and the filtered gyroscope data 509 may be superimposed for correction. In various examples, synthetic data described herein may include superimposed data described herein. This may remove the first level of wrong peaks/valleys. According to an embodiment, the correction of the accelerometer data (e.g., filtered accelerometer data 507) may include the correction of one or more erroneous peaks and valleys in the filtered accelerometer signal waveform of the accelerometer data.

According to an embodiment, for correcting the accelerometer data, the multi-sensor imposition component 201-2 may perform, using one or more filters, a signal refining process to smooth the signal waveform of the accelerometer data.

Thereafter, the multi-sensor imposition component 201-2 may determine a variation in a magnitude of the filtered accelerometer signal waveform of the accelerometer data with respect to a magnitude of the filtered gyroscope signal waveform of the filtered gyroscope data 509.

Thereafter, the multi-sensor imposition component 201-2 may adjust the determined variation based on the superimposition 511 of the accelerometer data over the gyroscope data. A signal waveform of the superimposed accelerometer data may be shown in block 513 (referring to FIG. 6) of FIG. 5. Further, the superimposed accelerometer data may be given by equation (2).

$$[ 1 ]$$

$$Super Imposed Data = \sqrt{\sqrt{{a}_{x}^{2} + {g}_{x}^{2}} + \sqrt{{a}_{y}^{2} + {g}_{y}^{2}} + \sqrt{{a}_{z}^{2} + {g}_{z}^{2}}} \qquad ( 2 )$$

$$where : {a}_{x} , {a}_{y} \& {a}_{z} : filtered accelerometer data ,$$

$${g}_{x} , {g}_{y} \& {g}_{z} \text{?} filtered gyroscape data$$

$$\text{?} \text{indicates text missing or illegible when filed}$$

According to an embodiment, the multi-sensor imposition component 201-2 may calculate a magnitude from the signal waveform of the superimposed accelerometer data. The calculated magnitude from the signal waveform of the superimposed accelerometer data may include a magnitude of the signal waveform of the superimposed accelerometer data, a magnitude of the signal waveform of the superimposed gyroscope data, and a running mean of the superimposed accelerometer data. The aforesaid calculated data may be further utilized by the dynamic thresholding component 201-3 for setting the dynamic thresholds.

According to some embodiments, the accelerometer data and the gyroscope data may be sent to the tilt detection component 201-4. The accelerometer data may be used for determining tilt/angle from a horizontal plane (e.g., posture of hand) between a vertical axis and a normal vector of the device. While walking or running, the angle of the electronic apparatus 100 or the hand posture of the user may be specific e.g., either in a pocket, or hand.

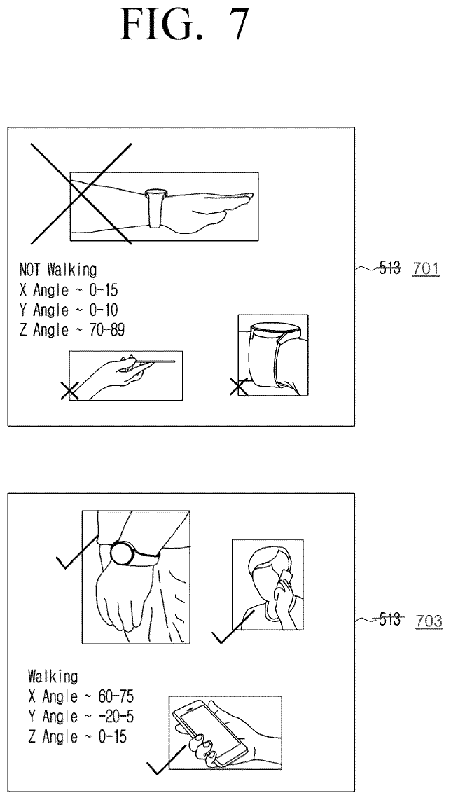

For instance, the device might not be kept horizontally when a user stops walking. FIG. 7 illustrates example postures of the user, according to an embodiment of the present disclosure. As can be seen at block 701, the shown postures cannot be the posture of the user during walking.

Further, block 703 shows the possible posture of the user during the walking. Accordingly, the posture can be detected by calculating the angles at which the electronic apparatus 100 is currently placed. The angle calculation can be done by the accelerometer sensor as the accelerometer leads to lesser current consumption and further helps to detect actual step counts.

According to an embodiment, the calculation of the device tilt angle as shown in block 703 will be explained. The tilt detection component 201-4 of the step sensing engine 201, may detect the tilt angle of the electronic apparatus 100 with respect to a horizontal plane based on the accelerometer data.

The tilt angle may indicate an angle between a vertical axis and a normal vector of the electronic apparatus 100. Thereafter, based on the detected tilt angle, the tilt detection component 201-4 may determine a posture of the electronic apparatus 100 during a walking activity of a user of the electronic apparatus 100. Thereafter, the tilt detection component 201-4 may determine whether the determined poster of the electronic apparatus 100 is one of a normal posture (e.g., as shown in block 703) or an abnormal posture (e.g., as shown at block 701) for the walking activity.

Thereafter, tilt detection component 201-4 may eliminate the determined posture of the electronic apparatus 100 based on a determination that the determined poster of the electronic apparatus is the abnormal posture. For example, at block 701, postures that belong to the abnormal posture may be eliminated.

According to an embodiment, the outputs of the multi-sensor imposition component 201-2 and the tilt detection component 201-4 may be fed to the dynamic thresholding component 201-3. Referring back to FIG. 3, at step 305, the dynamic thresholding component 201-3 may set a dynamic threshold value corresponding to the accelerometer data based on the magnitude of the signal waveform of the superimposed accelerometer data and the running mean of the superimposed accelerometer data. Accordingly, the peaks and valleys that are above and below the respective set dynamic threshold values may correspond to the actual steps.

After setting the dynamic threshold value, at step 307, the dynamic thresholding component 201-3 may detect a plurality of peaks and valleys in the accelerometer data based on the set dynamic threshold value. In particular, for detecting the plurality of peaks and valleys, the dynamic thresholding component 201-3 may calculate the magnitude of the signal waveform of the superimposed accelerometer data and then detects the plurality of peaks and valleys in the accelerometer data based on the calculated magnitude. Block 517 of FIG. 5 illustrates the dynamic thresholding operation.

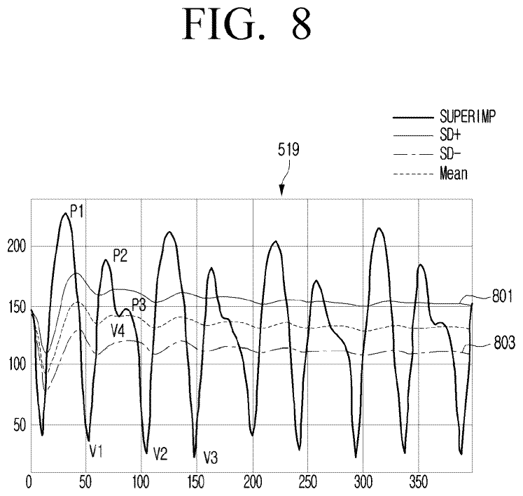

FIG. 8 illustrates an example of detecting the plurality of peaks and valleys in the accelerometer data, according to an embodiment of the present disclosure. FIG. 8 depicts the output 519 (referring to FIG. 8) of the dynamic thresholding component 201-3. As can be seen, P1 and P2 may be some of the valid peaks among other peaks (not shown) which are above an upper threshold value 801. Further, P3 may be one of the nonvalid peaks among other peaks (not shown) which is below the upper threshold value 801. Further, V1, V2 and V3 may be some of the valid valleys among other valleys (not shown) which are below a lower threshold value 803.

In the FIG. 8, the upper threshold value 801 may indicate a upper Standard Deviation (SD+) and the lower threshold value 803 may indicate a lower Standard Deviation (SD−).

Furthermore, V4 may be one of the nonvalid valleys among other valleys (not shown), which is above the lower threshold value 803. The output of the dynamic thresholding component 201-3 may be then fed to the fake step validation engine 203. The operation of the fake step validation engine 203 will be explained in the forthcoming paragraphs.

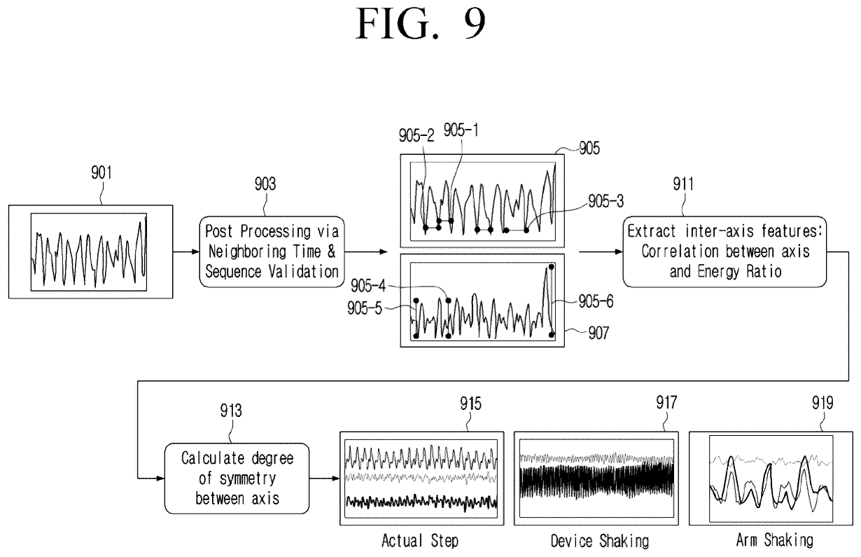

FIG. 9 illustrates an operation flow of the fake step validation engine 203, according to an embodiment of the present disclosure. As can be seen in FIG. 9, the output of the dynamic thresholding component 201-3 may be fed to the neighboring time/sequence validation component 203-1 of the fake step validation engine 203. The output may include the superimposed accelerometer data 901. Now the operation of the neighboring time/sequence validation component 203-1 will be explained through FIGS. 3, and 9 in the forthcoming paragraphs.

Referring back to FIG. 3, at step 309, the neighboring time/sequence validation component 203-1 of the fake step validation engine 203 with respect to human walking patterns may determine a signal pattern of the detected plurality of peaks and valleys corresponding to each axis of a plurality of axes of the electronic apparatus in a 3-Dimension (3D) coordinate system. In particular, the neighboring time/sequence validation component 203-1 may check if the peaks and valleys are within a reasonable time interval and sequence and satisfy certain time and order constraints that are characteristic of human walking patterns.

For example, while walking/running, the duration between any neighboring steps may be synchronized as any human cannot change the speed of walking/running at a sudden. Thus, the duration between any two consecutive steps might need to be in the range of its 5 previous neighboring steps. Thus, by considering such conditions, any random movements of the user which can be considered as steps but there are some random motions can be discarded. Amplitude of the step may be similar to the duration between consecutive steps.

The amplitude of any neighboring steps will be similar, and any random data may be eliminated from the basis dynamic amplitude threshold calculated via 5 previous steps. Thus, all other random movements which will give data similar to that of a step will be removed as they will not be consistent with their neighbors.

According to an embodiment, for determining the signal pattern, the neighboring time/sequence validation component 203-1 may analyze the superimposed accelerometer data 901 (e.g., the output) to check whether the detected plurality of peaks and valleys are within a reasonable time interval and whether a sequence of the detected plurality of peaks and valleys satisfies time and order constraints associated with human walking patterns. Further, the reasonable time interval may correspond to a time interval that is determined based on historical data associated with a walking activity of the user of the electronic apparatus 100.

Now based on a result of the analysis, the neighboring time/sequence validation component 203-1 may determine the signal pattern of the detected plurality of peaks and valleys. The result of the analysis can be seen at blocks 905 and 907. As can be seen, time intervals 905-1 and 905-2 may depict the synchronized steps as per the neighboring steps. Further, time interval 905-3 may depict non-synchronized steps based on the neighboring steps.

Further, amplitudes 905-4 and 905-5 may depict the synchronized steps as per the neighboring steps. Furthermore, amplitude 905-6 may depict not synchronized steps as per the neighboring steps. The operation post-processing via neighboring time and sequence validation by the neighboring time/sequence validation component 203-1 are shown in block 903.

After operation post-processing via neighboring time and sequence validation, at step 311, the inter-axis feature component 203-2 may measure one of a similarity or a difference in the determined signal patterns based on an extraction of a plurality of inter-axis features from the superimposed accelerometer data 901.

For doing so, the inter-axis feature component 203-2 may extract the inter-axis feature and determine a correlation between an axis and energy ratio in the superimposed accelerometer data. An operation of the inter-axis feature component 203-2 will be explained in the forthcoming paragraphs.

According to an embodiment, for measuring one of the similarities or the differences in the determined signal patterns, the inter-axis feature component 203-2, at first, may extract the inter-axis features including a correlation between axes and energy ratio of different axes among the plurality of axes of the electronic apparatus 100. According to an embodiment, the determined signal patterns may indicate a correlation between an axis and an energy ratio of different axes among the plurality of axes of the electronic apparatus 100.

Thereafter, the inter-axis feature component 203-2 may calculate a plurality of statistical features based on the magnitude of the signal waveform of the superimposed accelerometer data 901. In a non-limiting example, the plurality of statistical features may include at least a mean value of the signal waveform, a standard deviation in the signal waveform, and a skewness or kurtosis of the signal waveform.

Thereafter, based on the extracted inter-axis features and the calculated plurality of statistical features, the inter-axis feature component 203-2 may measure one of the similarity or the difference in the determined signal patterns. The operation of the extraction of the inter-axis features and the correlation between the axis and energy ratio is shown in block 911.

Subsequently, at step 313, the inter-axis feature component 203-2 may calculate a degree of symmetry between each axis of the plurality of axes based on one of the measured similarities or the measured difference in the determined signal patterns in block 913. During walking/running, asynchronous motion may occur in three directions, the with cross correlation between different axes being low.

In block 915, all three axes may have a low level of symmetry among each other. But for fake situations like device shaking, arm swing, etc., there may be motion in one or two directions. For example, block 917 may depict the device shaking. As illustrated in block 917, two of the axes may have a high level of symmetry among each other. Visually, X and Y axes may follow the same pattern.

Further, block 919 depicts an example of the arm swing. As shown in block 919, two of the axes may have the high level of symmetry among each other. Visually, X and Y axes may follow the same pattern. Accordingly, such a situation may cause synchronous fluctuation. Furthermore, the cross-correlation between different axes may be high. Further, the energy ratio is calculated using standard deviation which may depict fluctuation.

During walking/running, there may be motion in 3 directions, but there may be only motion in 1-2 directions in fake situations. Thus, the fluctuation of each axis may be different. The energy ratio may be higher in fake situations.

Subsequently, at step 317, the fake step validation engine 203 may eliminate the one or more fake step counts based on the calculated degree of symmetry. According to an embodiment, the elimination of the one or more fake step counts may correspond to a removal of one of a random movement performed by the user of the electronic apparatus 100 or a movement that generates signals similar to the walking and running activity of the user.

According to some embodiments, the fake step validation engine 203 may also perform another series of operations to eliminate the one or more fake step counts taken by the user. For example, firstly, the fake step validation engine 203 may calculate a magnitude of a set of peaks and valleys among the plurality of peaks and valleys in the accelerometer data.

The set of peaks and valleys may be associated with a set of steps previously taken by a user of the electronic apparatus 100. Second, the fake step validation engine 203 may determine, based on the calculated magnitude of the set of peaks and valleys, a peak threshold and a valley threshold corresponding to the accelerometer data.

Thirdly, the fake step validation engine 203 may determine a consistency between consecutive steps of the user of the electronic apparatus 100 based on each of the determined peak and valley thresholds. Lastly, the fake step validation engine 203 may eliminate the one or more fake step counts taken by the user of the electronic apparatus 100 based on the determining consistency.

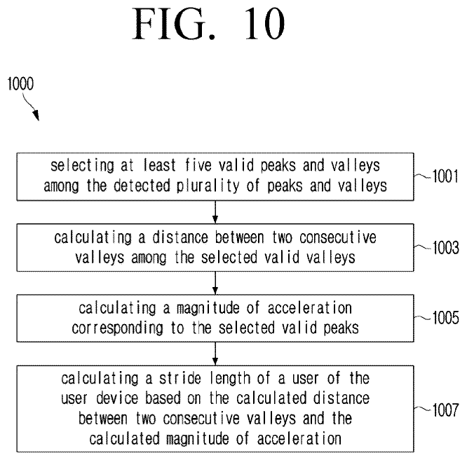

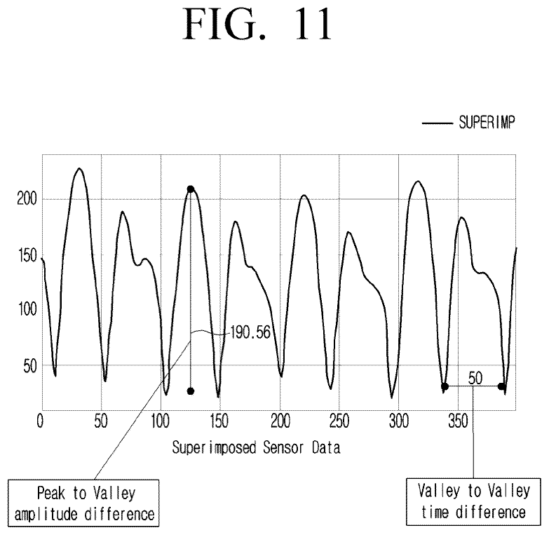

Now, referring back again to FIG. 2, the operation of the step length estimation module 205 will be explained in the forthcoming paragraphs in conjunction with FIGS. 10 and 11 of the drawings.

FIG. 10 illustrates a flow chart of a method 1000 implemented in the electronic apparatus 100 for calculating a stride length (e.g., step length) of the user of the electronic apparatus 100, according to an example embodiment of the present disclosure. The method 1000 may include a series of operation steps 1001 through 1007 performed by the step length estimation module 205.

At step 1001, the peak/valley selection 205-1 module of the step length estimation module 205 may select at least five valid peaks and valleys among the detected plurality of peaks and valleys from the superimposed accelerometer data.

At step 1003, the step length estimation module 205 may calculate a distance between two consecutive valleys among the selected valid valleys. In a non-limiting example, an example scenario depicting a calculated distance between two consecutive valleys is shown in FIG. 11 of the drawings. In particular, the distance between two consecutive valleys among the selected valid valleys may be calculated based on valley to valley time difference.

At step 1005, the step length estimation module 205 module may calculate a magnitude of acceleration corresponding to the selected valid peaks. In particular, for calculating the magnitude of acceleration corresponding to the selected valid peaks, the step length estimation module 205 may calculate a peak-to-valley and peak-to-peak amplitude distance. In a non-limiting example, an example scenario depicting a calculated magnitude of acceleration (e.g., peak to valley amplitude difference) corresponding to selected valid peaks is shown in FIG. 11 of the drawings.

In an embodiment of the present disclosure, the features calculated by the step length estimation module 205 may represent a change in acceleration, time taken, and frequency for each of the steps taken by the user of the electronic apparatus 100.

Thereafter, at step 1007, the stride length measurement 205-2 module of the step length estimation module 205 may calculate the stride length of the user of the electronic apparatus 100 based on the calculated distance between two consecutive valleys and the calculated magnitude of acceleration. In particular, in an embodiment of the present disclosure, the stride length measurement 205-2 module uses a pre-trained machine learning model where the aforesaid calculated features are provided as an input to the model and accordingly the pre-trained machine learning model outputs the accurate stride length of the user.

Once the stride length of the user is calculated using the method 1000, then the stride length measurement 205-2 module may further control a display screen of the electronic apparatus 100 to display a cumulative distance covered by the user of the electronic apparatus 100 during the walking or running activity based on the calculated stride length of the user.

In view of the above disclosed embodiments, the present disclosure provides various technical benefits and advantages that enhance the accuracy of the step count measurement as well as the user experience. For example, at least the method 300 may be performed to accurately differentiate between an actual user step and a random user motion or a random user movement and accordingly eliminates the random user motion or the random user movement while calculating a total distance travelled by the user during the walking or the running activity. Also, at least the disclosed method 300 can be performed to accurately determine the unit step length (e.g., stride length) of the user based on the user's way of walking/running.

Accordingly, at least the disclosed modules of the electronic apparatus 100 and the method 300 may provide personalized data to the user corresponding to the walking or running activity performed by the user.

While specific language has been used to describe the disclosure, any limitations arising on account of the same are not intended. As would be apparent to a person in the art, various working modifications may be made to the method to implement the embodiments of the disclosure as taught herein.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

Moreover, the actions of any flow diagram need not be implemented in the order shown nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

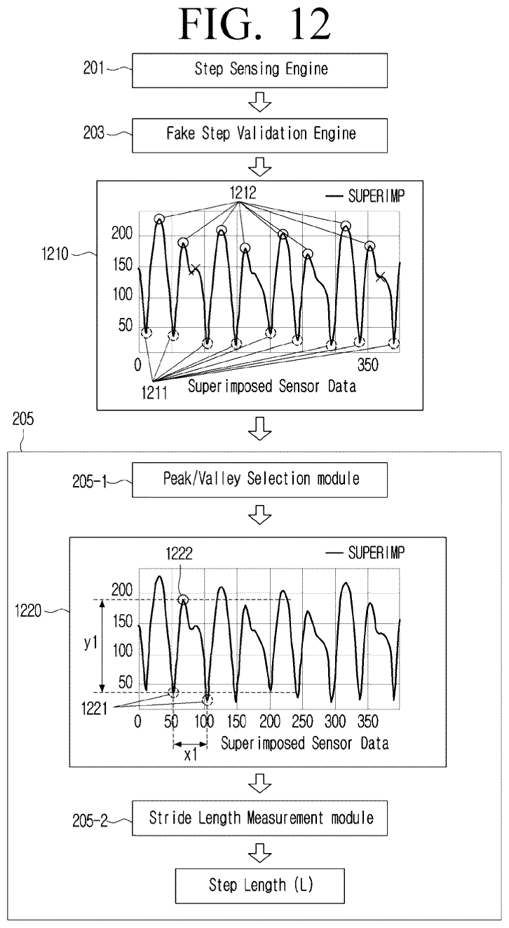

FIG. 12 illustrates an example of obtaining a step length.

The electronic apparatus 100 may include the step sensing engine 201, the fake step validation engine 203 and the step length estimation module 205. The modules of the step sensing engine 201, the components of the fake step validation engine 203, the modules of the step length estimation module 205 may be described in FIG. 2.

The electronic apparatus 100 may obtain the synthetic data through the step sensing engine 201. The electronic apparatus 100 may identify a plurality of critical points (e.g., peaks, valleys described herein) based on the synthetic data.

The electronic apparatus 100 may identify some fake steps in the plurality of critical points through the fake step validation engine 203. The electronic apparatus 100 may eliminate some fake step among the plurality of critical points in the synthetic data 1210 through the fake step validation engine 203.

The electronic apparatus 100 may identify a plurality of valleys 1211 and a plurality of peaks 1212 without the fake step based on the synthetic data 1210.

The electronic apparatus 100 may obtain the step length based on the synthetic data 1210 through the step length estimation module 205.

The step length estimation module 205 may include the peak/valley selection 205-1 module and the stride length measurement 205-2 module.

The electronic apparatus 100 may select at least one peak and at least one valley in the synthetic data 1220 through the peak/valley selection 205-1. The electronic apparatus 100 may identify at least three critical points based on the synthetic data 1220. The electronic apparatus 100 may select at least two valleys 1221 and at least one peak 1222 based on the at least three critical points.

The electronic apparatus 100 may obtain a stride length of a user based on the selected at least two valleys 1221 and the selected at least one peak 1222, through the stride length measurement 205-2 module.

The electronic apparatus 100 may obtain a difference value x1 between two consecutive valleys 1221 among the at least two valleys. The difference value x1 may indicate a time value. The electronic apparatus 100 may obtain the difference value x1 based on x-axis values of two consecutive valleys 1221.

The electronic apparatus 100 may obtain a magnitude y1 of acceleration corresponding to the at least one peak 1222. The electronic apparatus 100 may identify a first y-axis value of the at least one peak 1222. The electronic apparatus 100 may identify a second y-axis values based on y-axis value of at least one of the two consecutive valleys 1221. The electronic apparatus 100 may obtain the second y-axis by calculating an average value of y-axis values of the two consecutive valleys 1221. The electronic apparatus 100 may obtain the magnitude y1 of acceleration by calculating a difference value between the first y-axis value and the second y-axis value.

The electronic apparatus 100 may obtain a stride length of a user based on the difference value x1 between the two consecutive valleys 1221 and the magnitude y1 of acceleration.

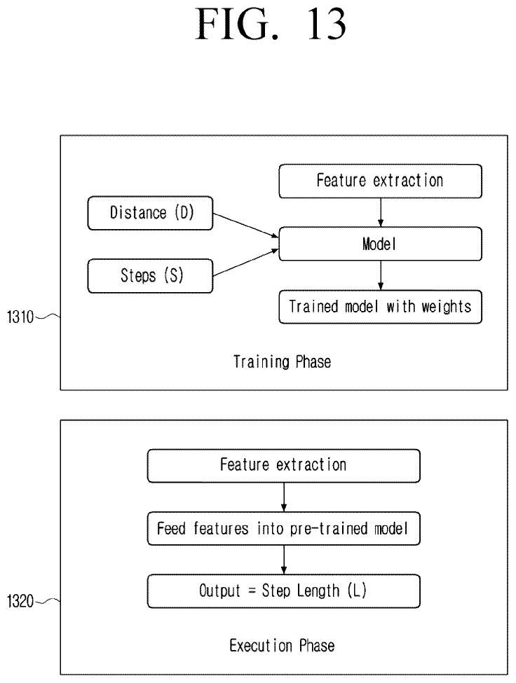

FIG. 13 illustrates an example of AI model.

The electronic apparatus 100 may store the AI model for calculating the step length of the user. The AI model may be trained based on the pre-determined training method.

According to an embodiment, the electronic apparatus 100 may train the AI model at the training phase 1310. The electronic apparatus 100 may obtaining training data for training the AI model. The training data may include motion data related to user's movement. The training data may be obtained from the first motion sensor and the second motion sensor.

The electronic apparatus 100 may train the AI model based a plurality of parameters. The plurality of parameters may include a distance factor and step factor. The electronic apparatus 100 may train the AI model with weights corresponding to the plurality of parameters.

The electronic apparatus 100 may consider a first weight corresponding to the distance factor and a second weight corresponding to the step factor. The electronic apparatus 100 may train the AI model based on the first weight and the second weight so that the AI model outputs step length of user's walking.

According to an embodiment, the electronic apparatus 100 may obtain the step length of the user's walking at execution phase 1320. The execution phase 1320 may be in real-time. The electronic apparatus 100 may obtain input data. The electronic apparatus 100 may extract feature from the input data.

The electronic apparatus 100 may input the feature into the pre-trained AI model in the embodiment. The electronic apparatus 100 may obtain output data including step length of the user's walking.

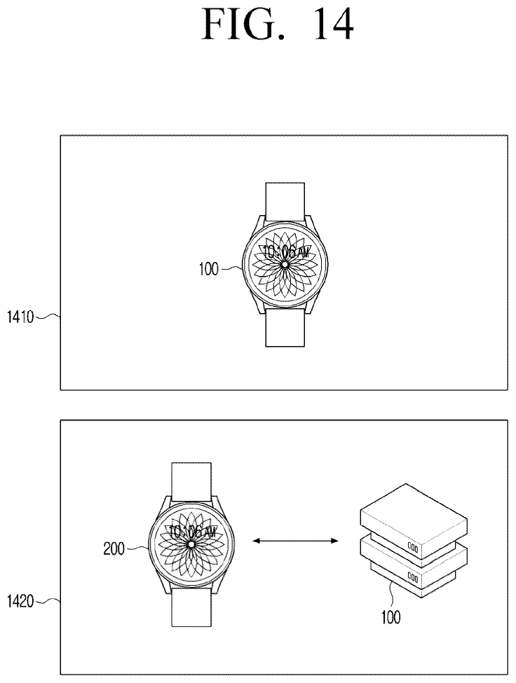

FIG. 14 illustrates examples of electronic apparatus.

According to an embodiment 1410, the electronic apparatus 100 may include at least one main motion sensor and at least one sub motion sensor.

The electronic apparatus 100 may include a memory 103 and at least one processor 101. The at least one processor 101 may obtain main motion data and sub motion data, obtain synthetic data by combining the main motion data and the sub motion data, obtain a threshold value based on a magnitude of a signal waveform of the synthetic data and a running mean of the synthetic data, identify a plurality of critical points in the synthetic data based on the threshold value, identify signal patterns, based on the plurality of critical points, in each axis of a plurality of axes in the main motion data of a 3-Dimension (3D) coordinate system, obtain at least one similarity in the signal patterns based on a plurality of inter-axis features extracted from the synthetic data, obtain a degree of symmetry between each axis of the plurality of axes based on the at least one similarity, and obtain a user's movement information based on the degree of symmetry and the synthetic data.

The electronic apparatus 100 may be described as an user device or terminal apparatus.

The main motion data may include accelerometer data.

The sub motion data may include gyroscope data. The threshold value may include dynamic threshold value.

The synthetic data may be described as superimposed accelerometer data.

The threshold value may be corresponding to the main motion data. The threshold value may be corresponding to the synthetic data.

The running mean may be described as running average, moving mean or moving average.

The critical point may include at least one of a peak point or a valley point. The critical point may be described as peaks and valleys.

The motion may be described as movement.

The signal pattern may be described as human walking pattern.

The similarity may be described as difference in the signal patterns.

The at least one similarity may include three similarities.

The at least one processor 101 may obtain three similarities based on the plurality of axes in the main motion data of the 3D coordinate system. For example, the 3D coordinate system may included x axis, y axis and z axis. Three similarities may include a first similarity between x axis and y axis, a second similarity between y axis and z axis, a three similarity between z axis and x axis.

The user's movement information may be described as user's motion information or user's body information. The user's information may be described as user's walking information or user's step information.

The main motion data may include information related to a measurement of an acceleration of the electronic apparatus 100 along the plurality of axes in the 3D coordinate system. The sub motion data may include information related to a change in a rotational angle of the electronic apparatus 100 in real-time along the plurality of axes in the 3D coordinate system.

The electronic apparatus 100 may include a sensor unit. The sensor unit may include at least one of sensors. The sensor unit may include at least one of a first sensor and a second sensor.

The first sensor may be an accelerometer sensor. The first sensor may sense (or obtain) a first data. The first data may include accelerometer data.

The second sensor may be gyroscope sensor. The second sensor may sense (or obtain) a second data. The second data may include gyroscope data.

The main motion data may be obtained from the at least one main motion sensor and the sub motion data is obtained from the at least one sub motion sensor.

The main motion data may include accelerometer data obtained from an accelerometer sensor. The sub motion data may include gyroscope data obtained from a gyroscope sensor. The signal patterns is identified according to a motion of the electronic apparatus 100 caused by a user's walking.

The at least one processor 101 may identify a plurality of user's steps based on the synthetic data, identify at least one fake step among the plurality of user's steps based on the degree of symmetry, and obtain the user's movement information by eliminating at least one fake step among the plurality of user's steps.

The degree of symmetry may be calculated based on the inter-axis similarity/difference.

The inter-axis features may be used for calculation of similarity/difference and in turn the extent of similarity/difference may be represented as the degree of symmetry.

The fake step may be described as fake step count.

The signal patterns may indicate a correlation between axis and energy ratio of different axes among the plurality of axes. The elimination of the at least one fake step may correspond to a removal of one of a random movement performed by a user or a movement that generates signals similar to walking and running activity of the user.

The at least one processor 101 may obtain a magnitude of a set of critical points among the plurality of critical points in the synthetic data, The set of critical points are associated with a set of steps previously taken by a user, identify a peak threshold and a valley threshold, based on the magnitude of the set of critical points, identify a consistency between consecutive steps of the user based on each of the peak threshold and the valley thresholds, and eliminating the at least one fake step based on the identify consistency.

The at least one processor 101 may identify a tilt angle of the electronic apparatus 100 with respect to a horizontal plane based on the main motion data. The tilt angle indicates an angle between a vertical axis and a normal vector of the electronic apparatus. The at least one processor 101 may identify a posture of the electronic apparatus 100 during a walking activity of a user, based on the tilt angle, identify whether the poster of the electronic apparatus 100 is one of a normal posture or an abnormal posture for the walking activity, and eliminating the posture of the electronic apparatus 100 based on a determination that the poster of the electronic apparatus 100 is the abnormal posture.

The at least one processor 101 may obtain the synthetic data by correcting the main motion data corresponds to a correction of one or more erroneous critical points in the signal waveform of the main motion data based on the sub motion data. The correcting the main motion data comprises performing, using one or more filters, a signal refining process to smooth the signal waveform of the main motion data, identifying a variation in magnitude of the signal waveform of the main motion data with respect to a magnitude of a signal waveform of the sub motion data, and adjusting the variation based on superimposition of the main motion data over the sub motion data.

The at least one processor 101 may obtain the magnitude of the signal waveform of the synthetic data, and identify the plurality of critical points in the synthetic data based on the magnitude.

The at least one processor 101 may extract, from the synthetic data, the plurality of inter-axis features including correlation between axes and energy ratio of different axes among the plurality of axes, obtain a plurality of statistical features based on the magnitude of the signal waveform of the synthetic data, obtain the at least one similarity in the signal patterns based on the plurality of inter-axis features and the plurality of statistical features.

The plurality of inter-axis features may obtained based on Correlation & Energy Ratio features.

The plurality of statistical features may include at least a mean value of the signal waveform, a standard deviation in the signal waveform, and a skewness and kurtosis of the signal waveform.

The at least one processor 101 may obtain the at least one similarity in the signal patterns based on how the inter-axis feature and the statistical feature.

The at least one processor 101 may analyze the synthetic data to check whether the plurality of critical points are within a reasonable time interval and whether a sequence of the plurality of critical points satisfies time and order constraints associated with human walking patterns, The reasonable time interval corresponds to a time interval that is determined based on historical data associated with a walking activity of a user, and identify the signal patterns based on a result of the analysis.

The at least one processor 101 may identify at least three critical points based on the synthetic data, select at least two valleys and at least one peak based on the at least three critical points, obtain a difference value between two consecutive valleys among the at least two valleys, obtain a magnitude of acceleration corresponding to the at least one peak, and obtain a stride length of a user based on the difference value between the two consecutive valleys and the magnitude of acceleration. The additional explanation is described in the FIG. 12.

According to an embodiment 1420, the electronic apparatus 100 may not include at least one main motion sensor and at least one sub motion sensor. The electronic apparatus 100 may receive the main motion data and the sub motion data from the external apparatus 200. The external apparatus 200 may include at least one main motion sensor and at least one sub motion sensor. The electronic apparatus 100 of the embodiment 1420 may perform the operations of the electronic apparatus 100 of the embodiment 1410. The electronic may be server for generating the user's movement information.

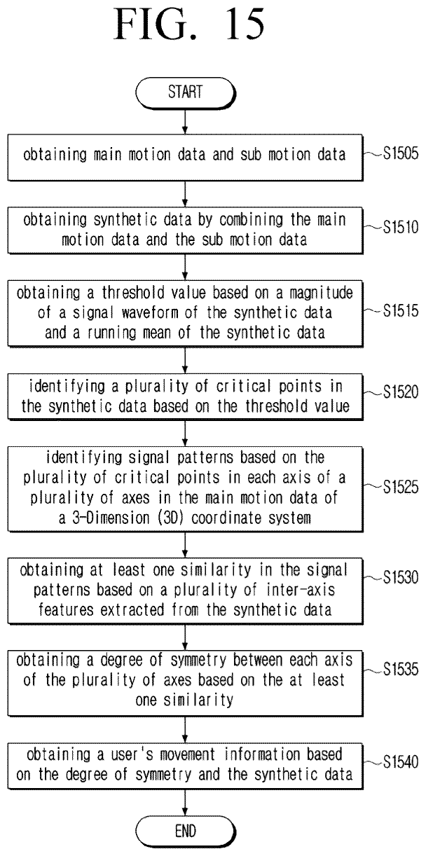

FIG. 15 illustrates a flow chart of a method for controlling the electronic apparatus.

According to an embodiment, a controlling method of an electronic apparatus, the method comprising obtaining main motion data and sub motion data (S1505), obtaining synthetic data by combining the main motion data and the sub motion data (S1510), obtaining a threshold value based on a magnitude of a signal waveform of the synthetic data and a running mean of the synthetic data (S1515), identifying a plurality of critical points in the synthetic data based on the threshold value (S1520), identifying signal patterns, based on the plurality of critical points, in each axis of a plurality of axes in the main motion data of a 3-Dimension (3D) coordinate system (S1525), obtaining at least one similarity in the signal patterns based on a plurality of inter-axis features extracted from the synthetic data (S1530), obtaining a degree of symmetry between each axis of the plurality of axes based on the at least one similarity (S1535), and obtaining a user's movement information based on the degree of symmetry and the synthetic data (S1540).

The main motion data may include information related to a measurement of an acceleration of the electronic apparatus along the plurality of axes in the 3D coordinate system. The sub motion data may include information related to a change in a rotational angle of the electronic apparatus in real-time along the plurality of axes in the 3D coordinate system.

The plurality of sensors may include at least one main motion sensor and at least one sub motion sensor. The main motion data may be obtained from the at least one main motion sensor and the sub motion data is obtained from the at least one sub motion sensor.

The main motion data may include accelerometer data obtained from an accelerometer sensor. The sub motion data may include gyroscope data obtained from a gyroscope sensor. The signal patterns may be identified according to a motion of the electronic apparatus caused by a user's walking.

Obtaining the user's movement may comprise (S1540) identifying a plurality of user's steps based on the synthetic data, identifying at least one fake step among the plurality of user's steps based on the degree of symmetry, and obtaining the user's movement information by eliminating at least one fake step among the plurality of user's steps.

The signal patterns may indicate a correlation between axis and energy ratio of different axes among the plurality of axes, The elimination of the at least one fake step may correspond to a removal of one of a random movement performed by a user or a movement that generates signals similar to walking and running activity of the user.

The method may further comprises obtaining a magnitude of a set of critical points among the plurality of critical points in the synthetic data. The set of critical points may be associated with a set of steps previously taken by a user. The method may further comprise identifying a peak threshold and a valley threshold, based on the magnitude of the set of critical points, identifying a consistency between consecutive steps of the user based on each of the peak threshold and the valley thresholds, and eliminating the at least one fake step based on the identifying consistency.

The method may further comprise identifying a tilt angle of the electronic apparatus with respect to a horizontal plane based on the main motion data. The tilt angle indicates an angle between a vertical axis and a normal vector of the electronic apparatus. The method may further comprise identifying a posture of the electronic apparatus during a walking activity of a user, based on the tilt angle, identifying whether the poster of the electronic apparatus is one of a normal posture or an abnormal posture for the walking activity, and eliminating the posture of the electronic apparatus based on a determination that the poster of the electronic apparatus is the abnormal posture.

Obtaining the synthetic data may comprise (S1510) obtaining the synthetic data by correcting the main motion data corresponds to a correction of one or more erroneous critical points in the signal waveform of the main motion data based on the sub motion data. Correcting the main motion data may comprise performing, using one or more filters, a signal refining process to smooth the signal waveform of the main motion data, identifying a variation in magnitude of the signal waveform of the main motion data with respect to a magnitude of a signal waveform of the sub motion data, and adjusting the variation based on superimposition of the main motion data over the sub motion data.

Identifying the plurality of critical points in the synthetic data may comprise (S1520) obtaining the magnitude of the signal waveform of the synthetic data, and identifying the plurality of critical points in the synthetic data based on the magnitude.

Obtaining the at least one similarity in the signal patterns may comprise (S1530) extracting, from the synthetic data, the plurality of inter-axis features including correlation between axes and energy ratio of different axes among the plurality of axes, obtaining a plurality of statistical features based on the magnitude of the signal waveform of the synthetic data, obtaining the at least one similarity in the signal patterns based on the plurality of inter-axis features and the plurality of statistical features.

The plurality of statistical features may include at least one of a mean value of the signal waveform, a standard deviation in the signal waveform, or a skewness and kurtosis of the signal waveform.

Identifying the signal patterns may comprise (S1525) analyzing the synthetic data to check whether the plurality of critical points are within a reasonable time interval and whether a sequence of the plurality of critical points satisfies time and order constraints associated with human walking patterns, The reasonable time interval may correspond to a time interval that is determined based on historical data associated with a walking activity of a user, and identifying the signal patterns based on a result of the analysis.

Obtaining a user's movement information may comprise (S1540) identifying at least two critical point based on the synthetic data, selecting at least two valleys and at least two peaks based on the at least two critical point, obtaining a distance between two consecutive valleys among the at least two valleys, obtaining a magnitude of acceleration corresponding to the at least two peaks, and obtaining a stride length of a user based on the distance between the two consecutive valleys and the magnitude of acceleration.

In one or more examples, the methods according to the aforementioned various embodiments of the disclosure may be implemented in forms of applications that may be installed on conventional electronic devices.

Also, the methods according to the aforementioned various embodiments of the disclosure may be implemented just with software upgrade, or hardware upgrade of conventional electronic apparatuses.

In addition, the aforementioned various embodiments of the disclosure may be performed through an embedded server provided on an electronic apparatus, or an external server of at least one of an electronic apparatus or a display apparatus.

In one or more examples, according to the various embodiments of the disclosure, the aforementioned various embodiments may be implemented as software including instructions stored in machine-readable storage media, which may be read by machines (e.g.: computers). The machines may refer to apparatuses that call instructions stored in a storage medium, and can operate according to the called instructions, and the apparatuses may include an electronic apparatus according to the aforementioned embodiments. In case an instruction is executed by a processor, the processor may perform a function corresponding to the instruction by itself, or by using other components under its control. An instruction may include a code that is generated or executed by a compiler or an interpreter. A storage medium that is readable by machines may be provided in the form of a non-transitory storage medium. Here, the term ‘non-transitory’ only means that a storage medium does not include signals, and is tangible, but does not indicate whether data is stored in the storage medium semi-permanently or temporarily.

According to the various embodiments of the disclosure, the methods according to the aforementioned various embodiments may be provided while being included in a computer program product. A computer program product may refer to a product, and it may be traded between a seller and a buyer. A computer program product may be distributed in the form of a storage medium that is readable by machines (e.g. a compact disc read only memory (CD-ROM)), or may be distributed on-line through an application store (e.g. Play Store™). In the case of on-line distribution, at least a portion of a computer program product may be stored in a storage medium such as the server of the manufacturer, the server of the application store, and the memory of the relay server at least temporarily, or may be generated temporarily.

In addition, each of the components (e.g.: a module or a program) according to the aforementioned various embodiments may comprise of a singular object or a plurality of objects. Among the aforementioned corresponding sub components, some sub components may be omitted, or other sub components may be further included in the various embodiments. Alternatively or additionally, some components (e.g.: a module or a program) may be integrated as an object, and perform functions that were performed by each of the components before integration identically or in a similar manner. Further, operations performed by a module, a program, or other components according to the various embodiments may be executed sequentially, in parallel, repetitively, or heuristically. Or, at least some of the operations may be executed in a different order or omitted, or other operations may be added.

While preferred embodiments of the disclosure have been shown and described, the disclosure is not limited to the aforementioned specific embodiments, and it is apparent that various modifications may be made by those having ordinary skill in the technical field to which the disclosure belongs, without departing from the gist of the disclosure as claimed by the appended claims. Further, it is intended that such modifications are not to be interpreted independently from the technical idea or prospect of the disclosure.

What is claimed is:

1. A method of controlling an electronic apparatus, the method comprising:
obtaining main motion data and sub motion data;
obtaining synthetic data by combining the main motion data and the sub motion data;
obtaining a threshold value based on a magnitude of a signal waveform of the synthetic data and a running mean of the synthetic data;
identifying a plurality of critical points in the synthetic data based on the threshold value;
identifying signal patterns, based on the plurality of critical points, in each axis of a plurality of axes in the main motion data of a 3-Dimension (3D) coordinate system;
obtaining at least one similarity in the signal patterns based on a plurality of inter-axis features extracted from the synthetic data;
obtaining a degree of symmetry between each axis of the plurality of axes based on the at least one similarity; and
obtaining movement information of a user based on the degree of symmetry and the synthetic data.

2. The method of claim 1, wherein the main motion data comprises information related to a measurement of an acceleration of the electronic apparatus along the plurality of axes in the 3D coordinate system, and
wherein the sub motion data comprises information related to a change in a rotational angle of the electronic apparatus in real-time along the plurality of axes in the 3D coordinate system.

3. The method of claim 1, wherein the main motion data is obtained from at least one main motion sensor and the sub motion data is obtained from at least one sub motion sensor.

4. The method of claim 1, wherein the main motion data comprise accelerometer data obtained from an accelerometer sensor,
wherein the sub motion data comprise gyroscope data obtained from a gyroscope sensor, and
wherein the signal patterns are identified according to a motion of the electronic apparatus caused by a movement of the user.

5. The method of claim 1, the obtaining the movement of the user comprises:
identifying steps of a plurality of users based on the synthetic data,
identifying at least one fake step from the steps of the plurality of users based on the degree of symmetry, and
obtaining the movement information of the user by eliminating the at least one fake step from the steps of the plurality of users.

6. The method of claim 5, wherein the signal patterns indicate a correlation between axis and energy ratio of different axes of the plurality of axes, and
wherein the elimination of the at least one fake step corresponds to a removal of a random movement performed by a user or a movement that generates first signals similar to second signals that are generated as a result of a walking and running activity of the user.

7. The method of claim 5, further comprising:
obtaining a magnitude of a set of critical points from the plurality of critical points in the synthetic data, wherein the set of critical points is associated with a set of steps previously taken by a user;
identifying a peak threshold and a valley threshold, based on the magnitude of the set of critical points;
identifying a consistency between consecutive steps of the user based on each of the peak threshold and the valley threshold; and
eliminating the at least one fake step based on identifying the consistency.

8. The method of claim 5, further comprising:
identifying a tilt angle of the electronic apparatus with respect to a horizontal plane based on the main motion data, wherein the tilt angle indicates an angle between a vertical axis and a normal vector of the electronic apparatus;
identifying a posture of the electronic apparatus while the user is walking based on the tilt angle;
identifying whether the posture of the electronic apparatus while the user is walking is a normal posture or an abnormal posture; and
eliminating the posture of the electronic apparatus based on identifying that the posture of the electronic apparatus is the abnormal posture.

9. The method of claim 1, wherein obtaining the synthetic data comprises:
correcting one or more erroneous critical points in the signal waveform of the main motion data based on the sub motion data, and
wherein the correcting the one or more erroneous critical points in the signal waveform of main motion data comprises:
performing, using one or more filters, a signal refining process to smooth the signal waveform of the main motion data;
identifying a variation in magnitude of the signal waveform of the main motion data with respect to a magnitude of a signal waveform of the sub motion data; and
adjusting the variation based on superimposition of the main motion data over the sub motion data.

10. The method of claim 1, wherein identifying the plurality of critical points in the synthetic data comprises:
obtaining the magnitude of the signal waveform of the synthetic data; and
identifying the plurality of critical points in the synthetic data based on the magnitude.

11. The method of claim 1, wherein obtaining the at least one similarity in the signal patterns comprises:
extracting, from the synthetic data, the plurality of inter-axis features including correlation between axes and energy ratio of different axes of the plurality of axes;
obtaining a plurality of statistical features based on the magnitude of the signal waveform of the synthetic data;
obtaining the at least one similarity in the signal patterns based on the plurality of inter-axis features and the plurality of statistical features.

12. The method as claimed in claim 11, wherein the plurality of statistical features includes at least one of a mean value of the signal waveform, a standard deviation in the signal waveform, or a skewness and kurtosis of the signal waveform.

13. The method of claim 1, wherein identifying the signal patterns comprises:
analyzing the synthetic data to check whether the plurality of critical points is within a reasonable time interval and whether a sequence of the plurality of critical points satisfies time and order constraints associated with human walking patterns, wherein the reasonable time interval corresponds to a time interval that is determined based on historical data associated with a walking activity of a user; and
identifying the signal patterns based on a result of the analysis of the synthetic data.

14. The method of claim 1, wherein obtaining a user's movement information comprises:
identifying at least three critical points based on the synthetic data;
selecting at least two valleys and at least one peak based on the at least three critical points;
obtaining a difference value between two consecutive valleys of the at least two valleys;
obtaining a magnitude of acceleration corresponding to the at least one peak; and
obtaining a stride length of a user based on the difference value between the two consecutive valleys and the magnitude of acceleration.

15. An electronic apparatus comprising:
at least one processor; and
memory storing instructions that, when executed by the at least one processor, cause the electronic apparatus to:
obtain main motion data and sub motion data,
obtain synthetic data by combining the main motion data and the sub motion data,
obtain a threshold value based on a magnitude of a signal waveform of the synthetic data and a running mean of the synthetic data,
identify a plurality of critical points in the synthetic data based on the threshold value,
identify signal patterns, based on the plurality of critical points, in each axis of a plurality of axes in the main motion data of a 3-Dimension (3D) coordinate system,
obtain at least one similarity in the signal patterns based on a plurality of inter-axis features extracted from the synthetic data,
obtain a degree of symmetry between each axis of the plurality of axes based on the at least one similarity, and
obtain movement information of a user based on the degree of symmetry and the synthetic data.
